(19) United States
(12) Patent Application Publication
(10) Pub. No.: US 2022/0208387 A1
(43) Pub. Date: Jun. 30, 2022

(54) MEDICAL INFORMATION PROCESSING APPARATUS

(71) Applicant: CANON MEDICAL SYSTEMS CORPORATION, Otawara-shi (JP)

(72) Inventors: Yuichi KAWABATA, Nasushiobara (JP); Katsuhiko FUJIMOTO, Saitama (JP); Fuminori FUJITA, Nasushiobara (JP); Michitaka SUGAWARA, Utsunomiya (JP); Mariko SHIBATA, Nasushiobara (JP)

(73) Assignee: CANON MEDICAL SYSTEMS CORPORATION, Otawara-shi (JP)

(21) Appl. No.: 17/643,011

(22) Filed: Dec. 7, 2021

(30) Foreign Application Priority Data
Dec. 8, 2020 (JP) 2020-203584
Dec. 6, 2021 (JP) 2021-197740

(51) Int. Cl. G16H 50/30; G16H 30/40

(52) U.S. Cl. CPC G16H 50/30; G16H 30/40

(57) ABSTRACT

A medical information processing apparatus comprises processing circuitry. The processing circuitry obtains from each of the patients entering a facility, identification information. The processing circuitry extracts when it is discovered that at least one of the patients has contracted a predetermined disease, an action taken in each of positions in the facility by a target patient who has contracted the disease, from action history information recording actions taken in the facility by each of the patients, on the basis of the identification information of the target patient. The processing circuitry calculates with respect to each of the positions in the facility, a first risk value of a risk imposed by the disease on a surrounding environment, on the basis of the actions of the target patient. The processing circuitry generates a risk map indicating the first risk values so as to be kept in association with the positions.

### CROSS-REFERENCE TO RELATED APPLICATIONS

This application is based upon and claims the benefit of priority from Japanese Patent Application No. 2020-203584, filed on Dec. 8, 2020; and Japanese Patent Application No. 2021-197740, filed on Dec. 6, 2021; the entire contents of all of which are incorporated herein by reference.

### FIELD

Embodiments described herein relate generally to a medical information processing apparatus.

### BACKGROUND

Conventionally, action history information of patients infected with an infectious disease (which hereinafter may be referred to as “infected patients”) is collected and managed, for the purpose of preventing the infectious disease and preventing the spread thereof. For example, a technique is proposed by which, without invading privacy of the infected patients, information such as a contact history is provided for people and medical facilities that wish to learn whether or not contact with the infected patients was made.

Further, when medical facilities such as hospitals and clinics admit a patient who is already known to be infected or a patient who is known to have been in contact with an infected patient, an attempt is made to prevent the spread of infection by, for example, separating the traffic flow of the patient from traffic flows of other patients, isolating the patient, and/or the like.

However, when an outpatient who is not known to be infected or to have been in contact is admitted before his/her being infected is learned through a medical test, for example, it is not possible to understand actions of the patient in the facility. Accordingly, there is a possibility that a countermeasure may need to be taken by closing down the facility or the like.

### BRIEF DESCRIPTION OF THE DRAWINGS

FIG. 1 is a drawing illustrating an exemplary configuration of a medical information processing system according to a first embodiment;

FIG. 2 is a drawing illustrating an exemplary configuration of a medical information processing apparatus according to the first embodiment;

FIG. 3 is a drawing illustrating an example of a risk value calculation table according to the first embodiment;

FIG. 4 is a drawing illustrating an example of a risk map according to the first embodiment;

FIG. 5 is a table illustrating an example of a disinfection plan according to the first embodiment;

FIG. 6 is a drawing illustrating the example of the disinfection plan according to the first embodiment;

FIG. 7 is a table illustrating an example of a risk map updating process according to the first embodiment;

FIG. 8 is a drawing illustrating the example of the risk map updating process according to the first embodiment;

FIG. 9 is an example of a flowchart illustrating an example of a risk map generating process performed by the medical information processing apparatus according to the first embodiment;

FIG. 10 is an example of a flowchart illustrating an example of a process related to disinfection carried out by the medical information processing apparatus according to the first embodiment;

FIG. 11 is a diagram illustrating an exemplary configuration of a medical information processing system according to a second embodiment;

FIG. 12 is a diagram illustrating an exemplary configuration of a controlling device included in a disinfection device according to the second embodiment;

FIG. 13 is a diagram illustrating an exemplary configuration of a medical information processing apparatus according to the second embodiment;

FIG. 14 is a drawing for explaining an example of a three-dimensional (3D) risk map projecting process according to the second embodiment;

FIG. 15 is a drawing for explaining another example of the 3D risk map projecting process according to the second embodiment;

FIG. 16 is a drawing for explaining yet another example of the 3D risk map projecting process according to the second embodiment; and

FIG. 17 is an example of a flowchart illustrating an example of a process related to disinfection carried out by the medical information processing apparatus according to the second embodiment.

### DETAILED DESCRIPTION

A medical information processing apparatus according to an embodiment includes a first obtaining unit, an extracting unit, a first calculating unit, a generating unit, and an output controlling unit. The first obtaining unit is configured to obtain, from each of the patients entering a facility, identification information capable of identifying the patient. The extracting unit is configured to extract, when it is discovered that at least one of the patients has contracted a predetermined disease, an action taken in each of positions in the facility by a target patient who has contracted the disease, from action history information recording actions taken in the facility by each of the patients identified with the identification information, on the basis of the identification information of the target patient. The first calculating unit is configured to calculate, with respect to each of the positions in the facility, a first risk value of a risk imposed by the disease on a surrounding environment, on the basis of the actions of the target patient extracted by the extracting unit. The generating unit is configured to generate a risk map indicating the first risk values of the positions calculated by the first calculating unit. The output controlling unit is configured to exercise control so as to output the risk map generated by the generating unit.

Exemplary embodiments of a medical information processing apparatus will be described below, with reference to the accompanying drawings.

### First Embodiment

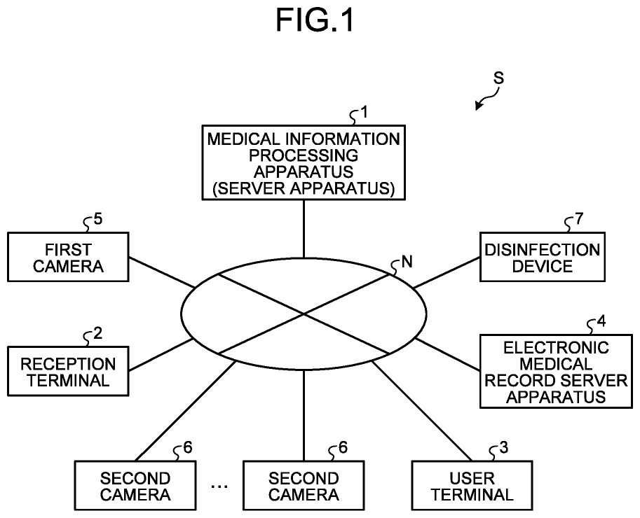

FIG. 1 is a drawing illustrating an exemplary configuration of a medical information processing system according to a first embodiment. As illustrated in FIG. 1, a medical information processing system S includes a medical information processing apparatus 1, a reception terminal 2, a user terminal 3, a first camera 5, second cameras 6, and a disinfection device 7.

The medical information processing apparatus 1, the reception terminal 2, the user terminal 3, an electronic medical record server apparatus 4, the first camera 5, the second cameras 6, and the disinfection device 7 are installed in a medical facility such as a hospital, for example, and are connected so as to be able to communicate with one another via a network N such as an intra-hospital network.

The reception terminal 2 is a computer installed at a reception counter of the medical facility, for example, and is configured to perform a reception process of diagnosis/treatment procedures for examined subjects (hereinafter, “patients”) according to an input made by reception staff. Further, the reception terminal 2 is configured to receive various types of information from the medical information processing apparatus 1. In this situation, the reception terminal 2 may be realized by using a mobile terminal (e.g., a notebook computer, a tablet computer, etc.).

The user terminal 3 is a computer provided at a nurse center or the like, a mobile terminal (e.g., a notebook computer, a tablet computer, etc.) held by medical staff, or the like. The user terminal 3 is configured to receive various types of information from the medical information processing apparatus 1.

The electronic medical record server apparatus 4 is a computer apparatus configured to manage electronic medical records. The electronic medical record server apparatus 4 is configured to transmit information (which hereinafter may be referred to as “test information”) including a result of a medical test related to a disease set in advance, to the medical information processing apparatus 1. The test information is, for example, information keeping the test result, a patient ID for identifying the patient, a face image of the patient, and the like in correspondence with one another. In the present embodiment, an example will be explained in which the disease in question is a predetermined infectious disease such as coronavirus disease 2019 (COVID-19). Further, in this situation, the test information of a patient who contracted the infectious disease includes a positive test result.

The first camera 5 is a thermal camera installed in the vicinity of the reception counter of the medical facility, for example. The first camera 5 is configured to take an image of the inside of the medical facility including patients visiting the medical facility and to analyze infrared rays emitted from objects. Further, the first camera 5 is configured to transmit, to the medical information processing apparatus 1, an image (which hereinafter may be referred to as “heat distribution image”) indicating a heat distribution as a diagram.

The image indicating the heat distribution taken by the first camera 5 is stored in a memory 120 (explained later) of the medical information processing apparatus 1, for example. In the present disclosure, examples of the image include a still picture, a video, and colors.

Accordingly, the medical information processing apparatus 1 is able to screen the patients visiting for a reception process of a diagnosis/treatment procedure, by using the body temperatures of the patients. As a result of detecting patients having a high risk for the infectious disease at the time of the reception process, the reception staff is also able to separate the traffic flows of the detected patients from those of the other patients, by guiding the detected patients to a high-fever outpatient department or the like. In this situation, the first camera 5 does not necessarily need to have the function of analyzing the infrared rays emitted from objects.

In this situation, for example, the medical information processing apparatus 1 is configured to screen the patients by analyzing the image received from the first camera 5 and understating whether or not each of the patients is taking an action of a high risk, such as frequent coughing.

The second cameras 6 are a plurality of cameras installed in various locations in the medical facility, for example. The second cameras 6 are configured to constantly take images of the inside of the medical facility and to transmit the taken images to the medical information processing apparatus 1 in a real-time manner. The images taken by the second cameras 6 are stored in the memory 120 of the medical information processing apparatus 1, for example.

Because the second cameras 6 are configured to constantly take the images of the inside of the medical facility (which hereinafter may be referred to as “inside facility images”), the medical information processing apparatus 1 is capable of tracking actions of patients and medical staff in the medical facility. Information obtained by tracking the actions of the patients and the medical staff is used for generating and updating a risk map. In this situation, the second cameras 6 may be 360-degree cameras each capable of taking images in all directions such as up-and-down and left-and-right directions at once.

The disinfection device 7 is configured to carry out a disinfection process in the medical facility, according to control exercised by the medical information processing apparatus 1. For example, the disinfection device 7 may be a self-propelled disinfection device having a function of emitting ultraviolet rays or a function of spraying a disinfectant. In this situation, the disinfection device 7 may be of any type, as long as the device is configured to carry out the disinfection process in the facility according to the control exercised by the medical information processing apparatus 1.

For example, it is acceptable to use a robot-like disinfection device, a disinfectant spray device installed on the ceiling or a wall in the medical facility, an ultraviolet ray emitting device installed on the ceiling or a wall in the medical facility, or the like. Alternatively, it is also acceptable to use a combination of two or more of the abovementioned devices as the disinfection device 7.

The medical information processing apparatus 1 is a dedicated or generic computer. For example, it is possible to realize the medical information processing apparatus 1 by using a server apparatus. The medical information processing apparatus 1 is configured to obtain the images of the examined subjects (e.g., the patients) and the medical staff from the first camera 5 and the second cameras 6.

On the basis of the images of the patients obtained from the first camera 5, the medical information processing apparatus 1 is configured to screen the patients. More specifically, the medical information processing apparatus 1 is configured to set a certain patient having a high risk for the infectious disease as an observation requiring person and to transmit information about the patient to the reception terminal 2 and the user terminal 3. By setting the patient having the high risk for the infectious disease as the observation requiring person, a user is able to immediately understand actions of the patient any time after the setting is established.

On the basis of the images of the patients and the like obtained from the second cameras 6, the medical information processing apparatus 1 is configured to extract the actions of the patient or the like having the high risk for the infectious disease. The medical information processing apparatus 1 is configured to generate the risk map on the basis of the actions of the patient or the like having the high risk for the infectious disease.

Further, the medical information processing apparatus 1 is configured to transmit the generated risk map to the user terminal 3. Furthermore, the medical information processing apparatus 1 is configured to generate a disinfection plan for the inside of the medical facility on the basis of the generated risk map. The medical information processing apparatus 1 is configured to carry out a disinfection process for the inside of the medical facility by controlling the disinfection device 7. Further, the medical information processing apparatus 1 is configured to update the risk map in conjunction with the execution of the disinfection process by the disinfection device 7.

Further, the medical information processing apparatus 1 is configured to receive information (e.g., a positive test result) used for judging contraction of the infectious disease, from the electronic medical record server apparatus 4 or the like.

For example, the medical information processing apparatus 1 may be realized by using a computer device such as a Personal Computer (PC) or a workstation. Alternatively, the medical information processing apparatus 1 may be realized with a plurality of computer devices (a cloud) using cloud computing.

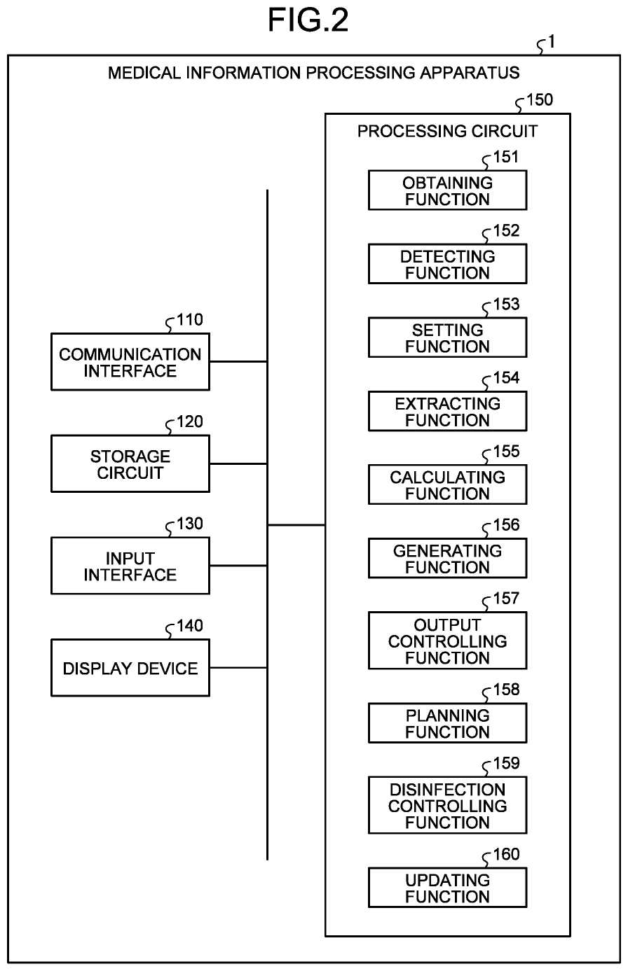

FIG. 2 is a drawing illustrating an exemplary configuration of the medical information processing apparatus 1 according to the first embodiment. As illustrated in FIG. 2, the medical information processing apparatus 1 includes a communication interface circuit 110, the memory 120, an input interface circuit 130, a display 140, and a processing circuit 150.

The communication interface circuit 110 is connected to the processing circuit 150 and is configured to control transfer of various types of data and communication between the medical information processing apparatus 1 and various systems.

More specifically, the communication interface circuit 110 is configured to receive information from various apparatuses and to output the received information to the processing circuit 150. For example, the communication interface circuit 110 is realized by using a network card, a network adaptor, a Network Interface Controller (NIC), or the like.

The memory 120 is connected to the processing circuit 150 and is configured to store therein various types of data. More specifically, the memory 120 is configured to store therein the heat distribution image taken by the first camera 5.

Further, in correspondence with each of the second cameras 6, the memory 120 is configured to store therein the inside facility image taken by the second camera 6. For example, the memory 120 may store an inside facility image taken by one of the second cameras 6 provided in a consultation room and an inside facility image taken by another one of the second cameras 6 provided in an examination room in mutually-different memory regions. Further, for example, the memory 120 may store identifiers each capable of identifying a different one of the second cameras 6 while being appended to a corresponding one of the inside facility images. In this situation, it is assumed that, as a result of appending a time code or the like to each of the inside facility images taken by the second cameras 6, it is possible to identify imaged times and to synchronize the images.

Further, the memory 120 is configured to store therein an action record of the patient set as the observation requiring person. Further, the memory 120 is configured to store therein a risk value calculation table (explained later). Further, the memory 120 is configured to store therein a camera position management table keeping correspondence relationships between each of the second cameras 6 and the installation location thereof.

For example, the memory 120 is realized by using a semiconductor memory element such as a Random Access Memory (RAM) or a flash memory, or a hard disk, an optical disk, or the like.

The input interface circuit 130 is connected to the processing circuit 150 and is configured to receive operations to input various types of instructions and various types of information from an operator. More specifically, the input interface circuit 130 is configured to convert the input operations received from the operator into electrical signals and to output the electrical signals to the processing circuit 150.

For example, the input interface circuit 130 is realized by using a trackball, a switch button, a mouse, a keyboard, a touchpad on which input operations can be performed by touching an operation surface thereof, a touch screen in which a display screen and a touchpad are integrally formed, a contactless input circuit using an optical sensor, an audio input circuit, and/or the like.

Further, the input interface circuit 130 does not necessarily have to include physical operation component parts such as a mouse, a keyboard, and/or the like. For instance, possible examples of the input interface circuit 130 include an electrical signal processing circuit configured to receive an electrical signal corresponding to an input operation from an external input device provided separately from the apparatus and to output the electrical signal to a controlling circuit.

The display 140 is connected to the processing circuit 150 and is configured to display various types of information and various types of images. More specifically, the display 140 is configured to convert data of the various types of information and the various types of images sent thereto from the processing circuit 150 into display-purpose electrical signals and to output the electrical signals. For example, the display 140 is realized by using a liquid crystal monitor, a Cathode Ray Tube (CRT) monitor, a touch panel, or the like.

The processing circuit 150 is configured to control constituent elements of the medical information processing apparatus 1 in accordance with the input operations received from the operator via the input interface circuit 130. More specifically, the processing circuit 150 is configured to store medical data output from the communication interface circuit 110 into the memory 120. Further, the processing circuit 150 is configured to read the medical data from the memory 120 and to cause the display 140 to display the read medical data. For example, the processing circuit 150 is realized by using a processor.

Further, the medical information processing apparatus 1 has various functions. More specifically, the medical information processing apparatus 1 includes an obtaining function 151, a detecting function 152, a setting function 153, an extracting function 154, a calculating function 155, a generating function 156, an output controlling function 157, a planning function 158, a disinfection controlling function 159, and an updating function 160.

In this situation, the obtaining function 151 is an example of the first obtaining unit. Also, the obtaining function 151 is an example of a second obtaining unit. The detecting function 152 is an example of a detecting unit. The extracting function 154 is an example of the extracting unit. The calculating function 155 is an example of the first calculating unit and a second calculating unit. The generating function 156 is an example of the generating unit. The output controlling function 157 is an example of the output controlling unit. The planning function 158 is an example of a planning unit. The disinfection controlling function 159 is an example of a disinfection controlling unit. The updating function 160 is an example of an updating unit.

The obtaining function 151 is configured to obtain various types of information. For example, from each of the patients entering the facility, the obtaining function 151 is configured to obtain patient identification information capable of identifying the patient. The patient identification information is an example of first identification information.

More specifically, the obtaining function 151 is configured to obtain, as the patient identification information of a patient, information (e.g., a face image of the patient) indicating appearance characteristics of the patient, from an image of the patient taken by one of the second cameras 6 installed at the reception counter, for example. The patient identification information obtained by the obtaining function 151 is stored in the memory 120 while being kept in association with the patient ID, for example.

In the present embodiment, the obtaining function 151 is configured to obtain the face image of the patient, as the patient identification information; however, not only the face image of the patient, the obtaining function 151 may obtain a whole body image of the patient as the patient identification information. With this arrangement, even when the patient cannot properly be identified from the face image alone due to a face mask or the like, it is possible to identify the patient by additionally using information about the physique, the clothing, and/or the like.

Further, for example, the obtaining function 151 is configured to obtain the inside facility images from each of the second cameras 6. The inside facility images obtained by the obtaining function 151 are stored in the memory 120, for example. Further, for example, the obtaining function 151 is configured to obtain the test information related to the infectious disease, from the electronic medical record server apparatus 4. The test information obtained by the obtaining function 151 is stored in the memory 120, for example.

On the basis of the states of the patients imaged by the first camera 5 and the second cameras 6, the detecting function 152 is configured to detect a patient having a high possibility of having contracted the infectious disease such as COVID-19. The first camera 5 and the second cameras 6 are examples of the sensor.

For example, the detecting function 152 is configured to detect a patient whose body temperature is equal to or higher than a threshold value (e.g., 37.5 degrees centigrade) in the heat distribution image taken by the first camera 5, as a patient having a high possibility of having contracted the infectious disease.

As another example, from a plurality of inside facility images taken by one of the second cameras 6 installed at the reception counter, the detecting function 152 is configured to detect a patient taking a predetermined action with a high risk such as frequent coughing, as a patient having a high possibility of having contracted the infectious disease.

In this situation, the face image of the patient detected as the patient having the high possibility of having contracted the infectious disease is stored in the memory 120, for example.

The setting function 153 is configured to establish various types of settings related to the medical information processing apparatus 1. For example, the setting function 153 is configured to set the patient having the high possibility of having contracted the infectious disease to be an observation requiring person.

More specifically, the setting function 153 is configured to set the patient detected by the detecting function 152 as a patient with a high risk of having contracted the infectious disease, to be the observation requiring person. Actions of the patient set by the setting function 153 as an observation requiring person are extracted by the extracting function 154 (explained later) in a real-time manner.

The images rendering the patient set as the observation requiring person and being extracted by the extracting function 154 in the real-time manner are arranged in a chronological order, for example, and are stored in the memory 120 as an action record while being kept in correspondence with the patient ID, separately from the inside facility images.

When it is discovered that at least one of the patients has contracted the infectious disease, the extracting function 154 is configured to extract, on the basis of the first identification information identifying a target patient who has contracted the infectious disease, actions taken by the target patient in various positions in the medical facility, from the action history information recording actions taken in the facility by each of the patients identified with the first identification information.

More specifically, when the obtaining function 151 obtains the test information including a positive test result related to the infectious disease from the electronic medical record server apparatus 4, the extracting function 154 is configured to extract an inside facility image rendering the patient, from among the plurality of inside facility images stored in the memory 120, by using the face image of the patient included in the test information as a key.

Further, upon receipt of an input to designate a patient ID from the user, the extracting function 154 may extract an inside facility image rendering the patient, from among the plurality of inside facility images stored in the memory 120, by using a face image of the patient corresponding to the patient ID, as a key.

After that, by analyzing the extracted inside facility image rendering the patient, the extracting function 154 is configured to recognize the actions taken by the patient in the various positions in the facility. The extracting function 154 is configured to arrange the recognized actions of the patient in a chronological order so as to be stored in the memory 120 as the action record of the patient while being kept in association with the patient ID of the patient.

In the present embodiment, it is assumed that having the positive test result means that the patient contracted the predetermined infectious disease. Further, the second cameras 6 are installed in the various locations in the medical facility and configured to constantly take the inside facility images. Accordingly, the plurality of inside facility images rendering each patient can be considered as information recording actions of each patient. In other words, the inside facility image can be considered as an example of the action history information.

On the basis of the actions of the target patient extracted by the extracting function 154, the calculating function 155 is configured to calculate, with respect to each of various positions in the medical facility, a risk value of a risk imposed by the infectious disease on the surrounding environment. The risk value is an example of the first risk value. More specifically, the calculating function 155 is configured to refer to the risk value calculation table stored in the memory 120 and to calculate a risk value with respect to each of the actions taken by the patient in the various positions in the facility.

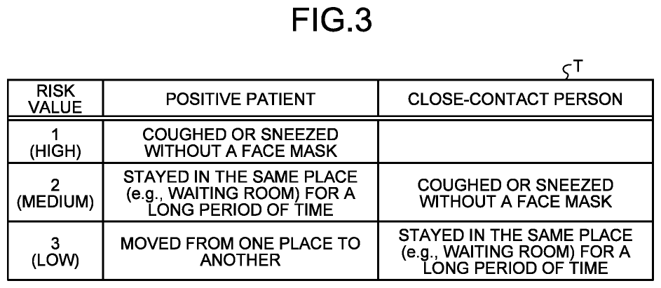

Next, the risk value calculation table will be explained, with reference to FIG. 3. FIG. 3 is a drawing illustrating an example of the risk value calculation table according to the first embodiment. A risk value calculation table T is a table keeping actions of patients in correspondence with risk values. For example, each of the risk values expresses, by using a numerical value, how much infection risk is caused by each action in the position where the action was taken.

In this regard, different infectious diseases have different routes of infection (e.g., droplet infection, airborne infection, and contact infection) and different levels of infectivity. Accordingly, the same action can cause a different infection risk depending on the infectious disease. For this reason, the risk value calculation table T is provided for each of different infectious diseases while taking routes of infection and infectivity thereof into consideration.

Further, in addition to the actions of infected patients, the risk value calculation table T stores therein actions of a close-contact person being another patient who has been in contact with an infected patient to an extent satisfying a predetermined condition, so as to be kept in correspondence with risk values. The reason is that the actions of the close-contact person are also considered to cause an infection risk.

In the risk value calculation table T in the example in FIG. 3, the action “coughed or sneezed without a face mask” of an infected patient is kept in correspondence with “risk value 3 (a high risk)”. Further, the action “stayed in the same place such as a waiting room for a long period of time” of an infected patient is kept in correspondence with “risk value 2 (a medium risk)”. Also, the action “moved from one place to another” of an infected patient is kept in correspondence with “risk value 1 (a low risk)”.

Further, the action “coughed or sneezed without a face mask” of a close-contact person is kept in correspondence with “risk value 2 (a medium risk)”. Also, the action “stayed in the same place such as a waiting room for a long period of time” of a close-contact person is kept in correspondence with “risk value 1”.

In the present example, more specifically, when the infected patient coughed without a face mask in a waiting room, for example, the calculating function 155 refers to the risk value calculation table T and calculates the value “3” kept in correspondence with the action of the infected patient having “coughed or sneezed without a face mask”, as a risk value of the waiting room.

In the example in FIG. 3, the risk values are on the three levels; however, the risk values do not necessarily have to be on three levels and may be on two levels or four or more levels, for example.

Further, with respect to a close-contact person, the calculating function 155 may calculate an individual risk value indicating a risk imposed by the infectious disease on the close-contact person. The individual risk value is an example of the second risk value. In this situation, the extracting function 154 is configured, at first, to specify the close-contact person on the basis of the action record of the infected patient. Subsequently, on the basis of the face image of the specified close-contact person, the extracting function 154 recognizes actions that were taken by the close-contact person in various positions in the facility.

The extracting function 154 is configured to arrange the recognized actions of the close-contact person in a chronological order so as to be stored in the memory 120 as an action record of the close-contact person, while being kept in association with a patient ID of the close-contact person. In this situation, because the face image and the patient ID of the close-contact person are stored in the memory 120 while being kept in correspondence with each other, it is possible to specify the patient ID of the close-contact person by obtaining the face image of the close-contact person.

Subsequently, on the basis of the action record of the infected patient and the action record of the close-contact person, the calculating function 155 is configured to calculate the individual risk value. For example, the calculating function 155 is configured to calculate the individual risk value on the basis of a degree of contact between the close-contact person and the infected patient.

In this situation, the calculating function 155 is configured to calculate the individual risk value of the close-contact person, by referring to an individual risk calculation table that keeps information indicating what type of contact was made between a close-contact person and an infected patient in correspondence with individual risk values. Further, when there are two or more close-contact people, the same process is performed with respect to each of the close-contact people.

Further, the calculating function 155 may calculate, with respect to each of various positions in the medical facility, a risk value of a risk imposed by the infectious disease on the surrounding environment, on the basis of the action record and the individual risk value of the close-contact person. As a result, the user is able to understand how much risk was imposed by the close-contact person on the surrounding environment.

Further, the calculating function 155 may calculate a severe symptom risk value of the close-contact person. In this situation, the severe symptom risk value is a value indicating a risk of symptoms related to the infectious disease becoming severe, as a result of being in contact with the infected patient.

In this situation, on the basis of the patient ID of the close-contact person, the calculating function 155 is configured to obtain patient information of the close-contact person from the electronic medical record server apparatus 4. In this situation, the patient information is information including the age and a health condition (whether or not he/she has diseases) of the patient, as well as a vaccination status of the patient against the infectious disease in question, or the like.

Further, on the basis of the individual risk value and the patient information, the calculating function 155 is configured to calculate the severe symptom risk value. For example, the calculating function 155 is configured to calculate the severe symptom risk value on the basis of a predetermined relation expression that makes the severe symptom risk value smaller when the individual risk value is smaller, when the age is lower, when the patient has no diseases, or when the patient has been vaccinated and that makes the severe symptom risk value larger when the individual risk value is larger, the age is higher, when the patient has a disease, or when the patient has not been vaccinated.

In this situation, the individual risk value and the severe symptom risk value may be output by the output controlling function 157 (explained later) to a display of the user terminal 3 or the like, while being kept in correspondence with the patient ID of the close-contact person or the like.

Because the individual risk value and the severe symptom risk value of the close-contact person are calculated and presented to the user, the user is able to easily establish a guideline as to how the close-contact person should be handled.

Returning to the description of FIG. 2, the generating function 156 is configured to generate a risk map indicating the risk values calculated by the calculating function 155 in association with various positions in the medical facility. For example, the risk map indicates a risk value of each of the various positions in the medical facility, in a corresponding position within a map of the medical facility.

More specifically, the generating function 156 is configured to generate the risk map in which the risk values calculated by the calculating function 155 are expressed in patterns so as to be indicated in the map of the medical facility in such a manner that the risk values are distinguishable from one another.

Further, the generating function 156 is configured to generate the risk map in which, as for a high risk area, what type of action was taken by the target patient in each position is also indicated in a corresponding position in the map where the action was taken, in addition to the risk value.

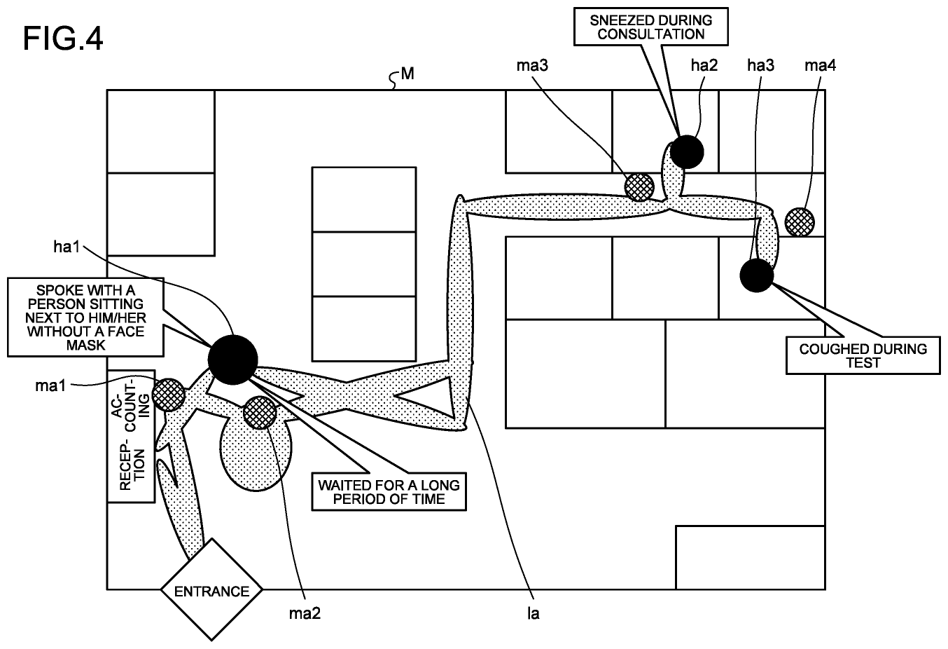

FIG. 4 is a drawing illustrating an example of the risk map generated by the generating function 156 according to the first embodiment. In the example in FIG. 4, a risk map M indicates areas (areas ha1 to ha3) each having risk value 3 with a solid black pattern, areas (areas ma1 to ma4) each having risk value 2 with a hatching pattern, and areas (an area 1a) having risk value 1 with a dot pattern.

Further, as for each of the areas having risk value 3, it is also indicated what type of action was taken by the infected patient in the area. For example, it is observed that, in the area ha1, the infected patient spoke with a person sitting next to him/her without a face mask. Also, in the area ha1, it is observed that the infected patient waited for a long period of time.

In addition, in the area ha2, it is observed that the infected patient sneezed during the consultation. Further, in the area ha3, it is observed that the infected patient was coughing during the test.

As explained above, the areas each having a high infection risk are indicated together with what types of actions were taken by the infected patient. As a result, medical staff or the like viewing the risk map is able to easily understand why the infection risks of those areas are high.

The output controlling function 157 is configured to control outputs of various types of information. For example, the output controlling function 157 is configured to exercise control so as to output the risk map generated by the generating function 156. More specifically, the output controlling function 157 is configured to exercise control so as to cause a display unit (e.g., a display) of the user terminal 3 to display the risk map generated by the generating function 156.

In this situation, the control exercised by the output controlling function 157 to output the risk map is not limited to the display control on the display of the user terminal 3. For example, the risk map may be output to a printer so as to be printed.

Further, when the detecting function 152 detects a patient having a high possibility of having contracted the infectious disease, the output controlling function 157 is configured to output a notification to notify the user of the detection. For example, in collaboration with the obtaining function 151 or the like, the output controlling function 157 is configured to obtain, from an electronic medical record, the memory 120 or the like, the patient ID and images or the like of the patient having the possibility of having been infected. Further, together with the image of the patient or the like, the output controlling function 157 is configured to transmit a message to the reception terminal 2 and to the user terminal 3 and to cause displays of these terminals to display the message, the message indicating that the patient having the possibility of having been infected is detected.

With these arrangements, on the basis of the content of the notification, the medical staff at the medical facility is able to take a countermeasure such as asking the patient having the high possibility of having contracted the infectious disease to wait in a place different from where the other patients are. Accordingly, it is possible to reduce an infection risk in the situation where the patient suspected to have the high possibility of having contracted the infectious disease actually contracted the infectious disease.

On the basis of the risk map generated by the generating function 156, the planning function 158 is configured to generate a disinfection plan defining a disinfection method of each of various positions in the medical facility. At first, the planning function 158 is configured to judge whether or not each of the various positions in the medical facility needs to be disinfected. Further, with respect to certain positions in the medical facility determined to require disinfection, the planning function 158 is configured to generate the disinfection plan defining methods of disinfection, time periods of disinfection, and a sequential order in which disinfection processes are to be performed.

More specifically, on the basis of the risk values in the risk map, the planning function 158 is configured to judge whether or not each of the areas requires disinfection. The planning function 158 is configured to generate the disinfection plan, by adjusting priority levels of the disinfection processes, how much time will be spent on the disinfection processes, and the like, with respect to the areas determined to require disinfection. For example, the disinfection plan defines in what sequential order, with what method, and how long the disinfection processes are to be performed by the disinfection device 7.

Even more specifically, for example, the planning function 158 is configured to generate the disinfection plan so as to prioritize the disinfection of the areas each having a higher risk value in the risk map. Further, for example, the planning function 158 is configured to generate the disinfection plan in such a manner that the higher risk value an area has in the risk map, the longer period of time will be spent in disinfecting the area.

In this situation, at the time of generating the disinfection plan, the planning function 158 may determine a disinfection method effective on the pathogen to be disinfected, as an element of the disinfection method. It is possible to specify the pathogen to be disinfected, on the basis of the test information that includes the positive test result related to the infectious disease and was obtained by the obtaining function 151.

For example, the disinfection method effective on the pathogen may be determined on the basis of a table keeping an effective disinfection method in correspondence with each of various types of pathogens. Further, when there are two or more effective disinfection methods, the planning function 158 is configured to determine the disinfection method on the basis of the time required to sufficiently carry out the disinfection, or the like.

In this situation, the planning function 158 may generate the disinfection plan by also taking work efficiency or the like into consideration. Further, the planning function 158 may generate the disinfection plan so as to include an instruction to set one or more Do Not Enter areas. In that situation, the setting function 153 is configured to set the Do Not Enter areas in the medical facility according to the setting instruction included in the disinfection plan generated by the planning function 158. Further, when the disinfection of the Do Not Enter areas has completed, the setting function 153 is configured to cancel the setting of the Do Not Enter areas.

Further, the planning function 158 may generate a plurality of disinfection plans to be presented to the user and may prompt the user to select one of the disinfection plans presented to the user.

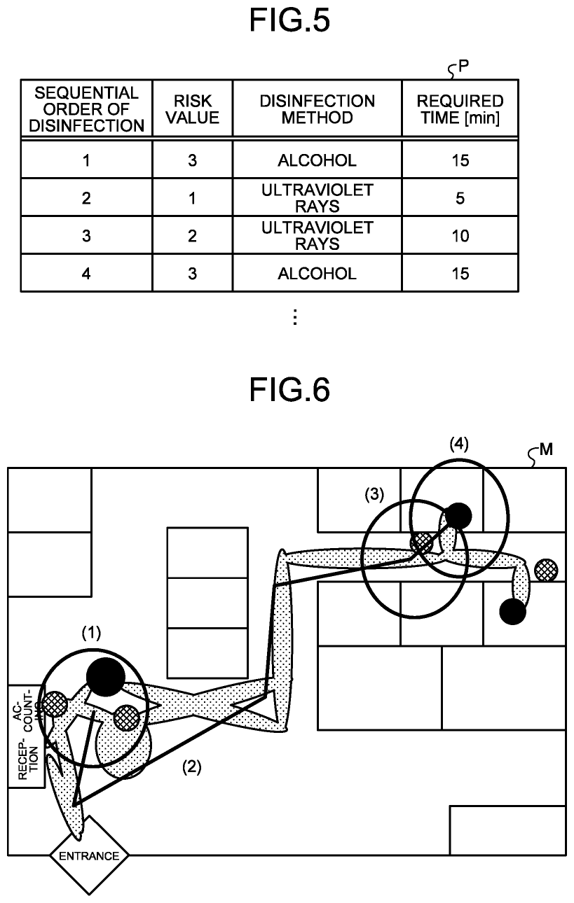

Next, the disinfection plan will be explained with reference to FIGS. 5 and 6. FIGS. 5 and 6 are a table and a drawing illustrating an example of the disinfection plan according to the first embodiment. A disinfection plan P indicates, with respect to certain areas in the risk map M that need to be disinfected, a sequential order of disinfection, risk values, disinfection methods, and required time periods. The sequential order of disinfection indicates in what sequential order the plurality of areas are to be disinfected.

In the example in FIGS. 5 and 6, the sequential order of disinfection is defined so that the disinfection processes are to be performed in the sequential order of: the inside of circled area (1), followed by the area around the part indicated with line (2), followed by the inside of circled area (3), followed by the inside of circled area (4), as indicated in the risk map M.

Further, as for the risk values, in the example in FIGS. 5 and 6, an average risk value of the inside of area (1) is 3, whereas an average risk value of area (2) is 1, an average risk value of the inside of area (3) is 2, and an average risk value of the inside of area (4) is 3.

Further, the disinfection methods are indicated so that the inside of area (1) is to be disinfected with alcohol, area (2) is to be disinfected with ultraviolet ray emission, the inside of area (3) is to be disinfected with ultraviolet ray emission, and the inside of area (4) is to be disinfected with alcohol.

Furthermore, the required time periods are indicated as follows: It will take 15 minutes to disinfect the inside of area (1); it will take 5 minutes to disinfect area (2); it will take 10 minutes to disinfect the inside of area (3); and it will take 15 minutes to disinfect the inside of area (4).

The disinfection controlling function 159 is configured to carry out the disinfection processes according to the disinfection plan generated by the planning function 158. More specifically, the disinfection controlling function 159 is configured to control the disinfection device 7 so as to disinfect the areas that need to be disinfected according to the sequential order, the methods, and the required time periods determined in the disinfection plan generated by the planning function 158.

Further, when there is an area which the disinfection device 7 is incapable of disinfecting (e.g., a place is too narrow for the disinfection device 7 to enter), the disinfection controlling function 159 is configured to cause the output controlling function 157 to output a notification thereof. More specifically, for example, the output controlling function 157 is configured to cause a speaker of the user terminal 3 to make an alert sound and to cause a display to display information prompting the user to manually carry out the disinfection.

In that situation, as for the incapability of the disinfection device 7 in carrying out the disinfection, the map may indicate, in advance, one or more areas where the automatic disinfection is impossible, so that the incapability is determined on the basis of the information. Alternatively, the disinfection device 7 may be provided with a camera or the like, so that the incapability is determined on the basis of images taken by the camera.

The updating function 160 is configured to update the risk map in conjunction with the disinfection processes carried out by the disinfection controlling function 159. More specifically, the updating function 160 is configured to perform, in a real-time manner, a process of decreasing the risk values of the areas in the risk map corresponding to the areas disinfected by the disinfection device 7 under the control of the disinfection controlling function 159.

Further, when the user manually carries out the disinfection, the obtaining function 151 is configured to obtain the face image of the user who carries out the disinfection, as user identification information. The user identification information is an example of second identification information. The extracting function 154 is configured to extract actions of the user who carries out the disinfection, from a plurality of inside facility images rendering the user.

Further, the updating function 160 is configured to perform, in a real-time manner, the process of decreasing the risk values of the areas in the risk map corresponding to the areas disinfected by the user.

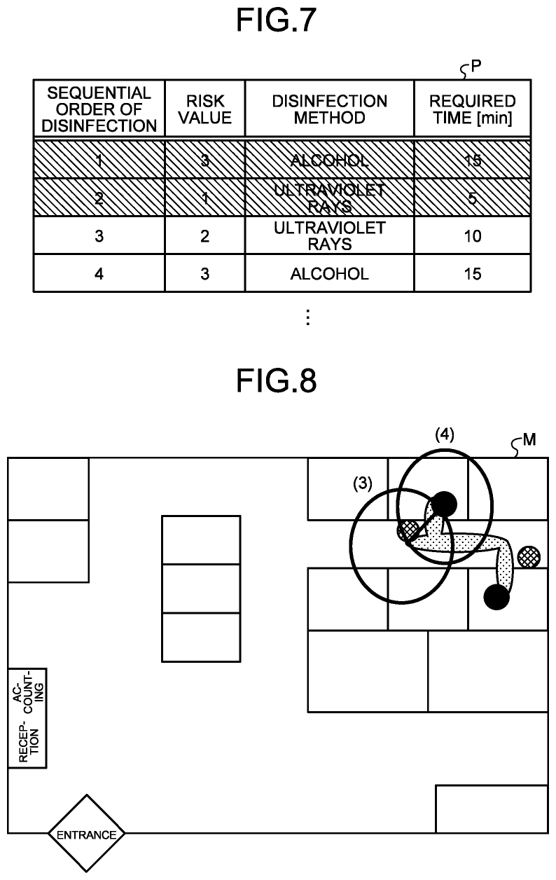

Next, the risk map updating process will be explained, with reference to FIGS. 5 to 8. FIGS. 7 and 8 are a table and a drawing illustrating an example of the risk map updating process according to the first embodiment. For example, let us discuss an example in which the planning function 158 generates the disinfection plan P in FIG. 5, so that the disinfection controlling function 159 controls the disinfection device 7 according to the disinfection plan P so as to carry out the disinfection up to number 2 in the sequential order of disinfection.

In this situation, the updating function 160 performs the process of decreasing the risk values of the inside of area (1) and area (2) in the risk map M in FIG. 6. Accordingly, as illustrated in FIGS. 7 and 8, the inside of area (1) and area (2) in the risk map M that have been disinfected are updated to be in the state of having no infection risk.

In the situation where the inside of area (1) and area (2) in the risk map M in FIG. 6 had been set as Do Not Enter areas by the setting function 153, the setting function 153 cancels the setting of the Do Not Enter areas for the inside of areas (1) and area (2) in the risk map M, when the disinfection controlling function 159 finishes disinfecting the areas corresponding to the inside of area (1) and area (2).

Further, when all the disinfection processes determined in the disinfection plan generated by the planning function 158 have been completed, the updating function 160 is configured to cause the output controlling function 157 to output a notification informing the user that the disinfection processes are completed. More specifically, for example, the output controlling function 157 is configured to cause a display of the user terminal 3 to display information indicating that the disinfection processes are completed.

Because the updating function 160 is configured to update the risk map in the real-time manner, the user is able to immediately understand which places have already been disinfected and is thus able to easily guide patients or the like.

The term “processor” used in the above explanations denotes, for example, a Central Processing Unit (CPU), a Graphics Processing Unit (GPU), or a circuit such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]).

When the processor is a CPU, for example, the processor realizes the functions by reading and executing the programs saved in the memory 120. In contrast, when the processor is an ASIC, for example, instead of the programs being saved in the memory 120, the functions are directly incorporated in the circuit of the processor as a logic circuit.

Further, the processors of the present embodiments do not each necessarily have to be structured as a single circuit. It is also acceptable to structure one processor by combining together a plurality of independent circuits, so as to realize the functions thereof. Further, it is also acceptable to integrate two or more of the constituent elements in any of the drawings into a single processor, so as to realize the functions thereof.

In this situation, the programs executed by the processors are provided as being incorporated in advance in a Read-Only Memory (ROM), a memory, or the like. Alternatively, the programs may be provided as being recorded on a computer-readable memory medium such as a Compact Disk (CD)-ROM, a Flexible Disk (FD), a CD-Recordable (CD-R), a Digital Versatile Disk (DVD), or the like in a file that is in an installable or executable format for the abovementioned devices.

Further, the programs may be stored in a computer connected to a network such as the Internet, so as to be provided or distributed as being downloaded via the network. For example, the programs are structured with modules including the functional units described above.

In the actual hardware, as a result of a CPU reading and executing the programs from the memory medium such as a ROM, the modules are loaded into a main memory so as to be generated in the main memory.

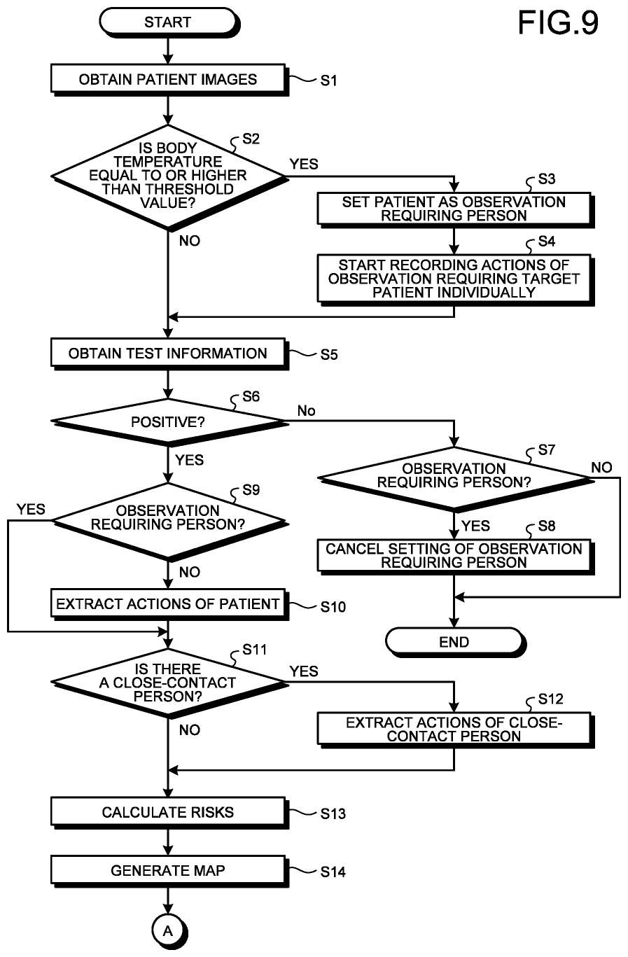

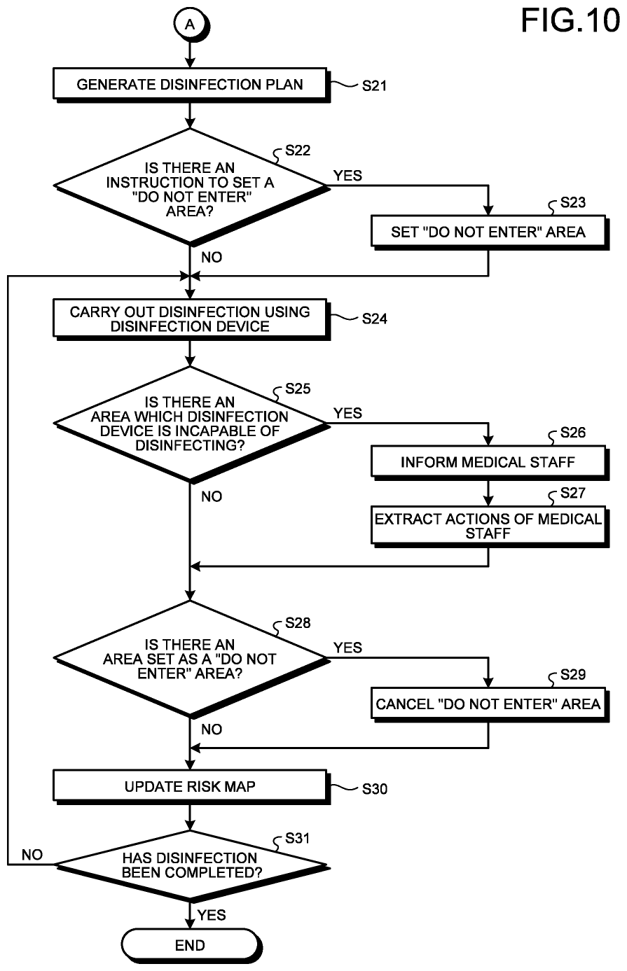

Next, processes performed by the medical information processing apparatus 1 according to the first embodiment will be explained, with reference to FIGS. 9 and 10. FIG. 9 is a flowchart illustrating an example of the risk map generating process performed by the medical information processing apparatus 1 according to the first embodiment.

The obtaining function 151 obtains the face image of a patient from among the inside facility images taken by the second cameras 6 at the time of reception (step S1). The face image taken by the obtaining function 151 is stored into the memory 120 while being kept in correspondence with the patient ID.

At the same time as step S1, the detecting function 152 analyzes a heat distribution image of the patient taken by the first camera 5 at the time of reception and checks to see whether or not the body temperature of the patient is equal to or higher than the threshold value (step S2). When the body temperature of the patient is lower than the threshold value (step S2: No), the process proceeds to step S5.

On the contrary, when the body temperature of the patient is equal to or higher than the threshold value (step S2: Yes), the detecting function 152 detects the patient as a patient with a high risk of having contracted the infectious disease. Further, the setting function 153 sets the patient whose body temperature is equal to or higher than the threshold value as an observation requiring person (step S3). The extracting function 154 extracts actions of the patient being the observation requiring person in a real-time manner and causes the memory 120 to start recording the actions of the patient, separately from the inside facility images (step S4).

The obtaining function 151 obtains test information from the electronic medical record server apparatus 4 (step S5). The setting function 153 checks to see whether or not a positive test result related to the infectious disease is included in the test information obtained by the obtaining function 151 (Step S6).

When no positive test result is included (step S6: No), the setting function 153 checks to see whether or not the patient is an observation requiring person (step S7). When the patient is not an observation requiring person (step S7: No), the present process is ended. On the contrary, when the patient is an observation requiring person (step S7: Yes), the setting of the patient being an observation requiring person is cancelled, and the present process is ended (step S8).

At step S6, when a positive test result is included (step S6: Yes), the extracting function 154 checks to see whether or not the patient is an observation requiring person (step S9). When the patient is an observation requiring person (step S9: Yes), the process proceeds to step S11.

On the contrary, when the patient is not an observation requiring person (step S9: No), the extracting function 154 extracts actions of the patient from the plurality of inside facility images stored in the memory 120 (step S10). The extracting function 154 causes the memory 120 to store therein the extracted actions of the patient, as an action record of the patient.

On the basis of the action record of the patient, the extracting function 154 checks to see whether or not there is a close-contact person of the patient (step S11). When there is no close-contact person (step S11: No), the process proceeds to step S13.

On the contrary, when there is a close-contact person (step S11: Yes), the extracting function 154 extracts actions of the close-contact person from the plurality of inside facility images stored in the memory 120 (step S12). The extracting function 154 causes the memory 120 to store therein the extracted actions of the close-contact person, as an action record of the close-contact person.

The calculating function 155 refers to the risk value calculation table T stored in the memory 120 and the action record of the patient having the positive test result related to the infectious disease and calculates risk values of the actions of the patient in various positions in the facility (step S13). When there is a close-contact person of the patient, the calculating function 155 also calculates risk values of the actions of the close contact in various positions in the facility.

The generating function 156 generates a risk map indicating the risk values of the various positions in the medical facility calculated by the calculating function 155 and proceeds to disinfection process A (step S14). The risk map generated by the generating function 156 is output to the user terminal 3 or the like by the output controlling function 157.

Next, FIG. 10 is a flowchart illustrating an example of a process related to the disinfection carried out by the medical information processing apparatus 1 according to the first embodiment.

The planning function 158 generates a disinfection plan on the basis of the risk map generated by the generating function 156 (step S21). The setting function 153 checks to see whether or not the disinfection plan generated by the planning function 158 includes an instruction to set Do Not Enter areas (step S22). When no instruction to set Do Not Enter areas is included (step S22: No), the process proceeds to step S24.

On the contrary, when an instruction to set Do Not Enter areas is included (step S22: Yes), the setting function 153 sets, as the Do Not Enter areas, certain areas in the medical facility designated in the disinfection plan generated by the planning function 158 (step S23). The setting of the Do Not Enter areas is reflected in the risk map by the updating function 160.

The disinfection controlling function 159 controls the disinfection device 7 so as to start disinfecting the areas that need to be disinfected, according to the disinfection plan generated by the planning function 158 (step S24). The disinfection controlling function 159 checks to see whether or not the areas that need to be disinfected include one or more areas which the disinfection device 7 is incapable of disinfecting (step S25). When there is no area which the disinfection device 7 is incapable of disinfecting (step S25: No), the process proceeds to step S28.

On the contrary, when there is at least one area which the disinfection device 7 is incapable of disinfecting (step S25: Yes), the disinfection controlling function 159 causes the output controlling function 157 to issue a notification prompting the user to manually carry out the disinfection because there is at least one area which the disinfection device 7 is incapable of disinfecting (e.g., by causing a speaker of the user terminal 3 to make an alert sound, in addition to causing a display to display information prompting the manual disinfection of the specific area) (step S26).

When the user manually carries out the disinfection, the obtaining function 151 obtains a face image of the user who carries out the disinfection. Further, on the basis of the face image of the user, the extracting function 154 extracts actions of the user from the plurality of inside facility images (step S27). Further, on the basis of the actions of the user, the disinfection controlling function 159 recognizes the execution of the disinfection.

The setting function 153 checks to see whether or not the areas that have been disinfected include one or more areas that are set as Do Not Enter areas (step S28). When there is no area set as a Do Not Enter area (step S28: No), the process proceeds to step S30.

On the contrary, when there is at least one area set as a Do Not Enter area (step S28: Yes), the setting function 153 cancels the setting of the Do Not Enter area among the areas that have been disinfected (step S29). The cancellation of the setting of the Do Not Enter area is reflected in the risk map by the updating function 160.

The updating function 160 the updating function 160 updates the risk map by decreasing the risk value of the areas in the risk map corresponding to the areas that have been disinfected by the disinfection controlling function 159 (step S30). Further, the updating function 160 checks to see whether or not the disinfection determined in the disinfection plan has been completed (step S31).

When the disinfection has not been completed (step S31: No), the process proceeds to step S21. On the contrary, when the disinfection has been completed (step S31: No), the output controlling function 157 outputs a notification to notify the user that the disinfection has been completed (e.g., by causing a display of the user terminal 3 to display information indicating that the disinfection has been completed), and the present process is thus ended.

The medical information processing apparatus 1 according to the first embodiment described above includes the obtaining function 151 serving as an obtaining unit, the extracting function 154 serving as the extracting unit, the calculating function 155 serving as a calculating unit, the generating function 156 serving as the generating unit, and the output controlling function 157 serving as the output controlling unit.

From each of the patients entering the medical facility, the obtaining function 151 is configured to obtain the identification information of the patient as the patient identification information capable of identifying the patient. When it is discovered that at least one of the patients has contracted the predetermined disease, the extracting function 154 is configured, on the basis of the identification information of the target patient who has contracted the disease, to extract the actions taken by the target patient in each of the various positions in the medical facility, from the action history information recording the actions taken in the medical facility by each of the patients identified with the identification information. The calculating function 155 is configured to calculate, with respect to each of the positions in the medical facility, the risk value of the risk imposed by the disease on the surrounding environment, on the basis of the actions of the target patient extracted by the extracting function 154. The generating function 156 is configured to generate the risk map indicating the risk values calculated by the calculating function 155 so as to be kept in association with the positions in the medical facility. The output controlling function 157 is configured to exercise control so as to output the risk map generated by the generating function 156.

As a result, when it is discovered that the patient contracted the infectious disease, the risk map visualizes the actions of the patient and the degrees of infection risks in the range of actions of the patient. Because the actions of the patient and the degrees of infection risks are visualized, viewers of the risk map are able to easily understand the actions of the patient. Consequently, by using the medical information processing apparatus 1 according to the present embodiment, it is possible to understand the actions taken in the facility by the patient who was found to have contracted the infectious disease.

Further, because it is possible to understand the actions taken in the facility by the patient who was found to have contracted the infectious disease, it is considered to be easier for the medical staff to plan a countermeasure for the situation where there is a person infected by the infectious disease.

Further, when there is an infected person, by disclosing the risk map visualizing the actions of the infected person and the degrees of infection risks, to patients and the like who are suspected to have been in contact with the infected person, it is possible to relieve anxiety of some patients who can confirm that no contact was made with the infected person. Conversely, for other patients who can confirm that there is a possibility that he/she may have been in contact with the infected person, it is possible to urge some actions that will reduce the infection risk by, for example, taking actions on the assumption that he/she is also infected.

### Second Embodiment

A second embodiment is based on the first embodiment. In the second embodiment, the medical information processing apparatus 1 is configured to generate a three-dimensional risk map (which hereinafter may be referred to as “3D risk map”) and to project the 3D risk map inside the facility by using a projector. In the following sections, differences between the second embodiment and the first embodiment will be explained, and duplicate explanations will be omitted as appropriate. Further, some of the elements that are the same as those in the first embodiment will be referred to by using the same reference characters, and the explanations thereof will be omitted.

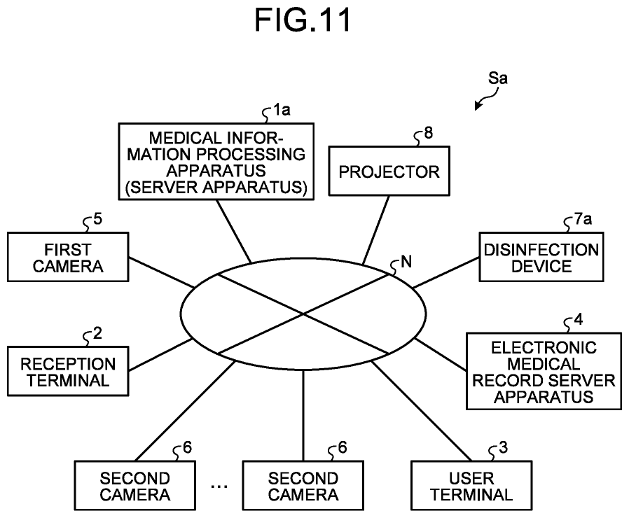

FIG. 11 is a diagram illustrating an exemplary configuration of a medical information processing system according to the second embodiment. As illustrated in FIG. 11, a medical information processing system Sa includes a medical information processing apparatus 1a, the reception terminal 2, the user terminal 3, the first camera 5, the second cameras 6, a disinfection device 7a, and a projector 8.

To begin with, the disinfection device 7a will be explained. The disinfection device 7a is a device used by a user to manually carry out disinfection. For example, the disinfection device 7a may be a disinfection mop. The disinfection mop includes a tank for storing therein a disinfectant fluid and an application part provided with tassels, non-woven fabric, or the like used for applying the disinfectant fluid supplied from the tank. The tank is configured to automatically supply the application part with the disinfectant fluid in an amount necessary for the disinfection. By wiping locations to be disinfected with the disinfection mop, the user is able to apply the disinfectant fluid to the locations.

The amount of the disinfectant fluid to be supplied may be adjusted under control of a controlling device 70 (explained later). Alternatively, for example, the adjustment may be made by a mechanism configured to adjust the supplied amount by changing the number of times or the pressure level in the process of pressing the disinfection mop against the floor or the like.

Further, for example, the disinfection device 7a may be a disinfection sprayer configured to spray a disinfectant. The disinfection sprayer is a device configured to spray the disinfectant over objects to be disinfected by using pressure or the like. The disinfection sprayer includes a tank for storing therein the disinfectant fluid and a nozzle for spraying the disinfectant fluid stored in the tank. By directing the nozzle at locations to be disinfected and spraying the disinfectant thereon, the user is able to spray the disinfectant fluid over the locations.

In this situation, the amount of the disinfectant fluid to be sprayed may be adjusted under control of the controlling device 70 (explained later). Alternatively, for example, the adjustment may be made by a mechanism configured to adjust the sprayed amount by changing the number of times or the pressure level in the process of operating a lever used for spraying the disinfectant.

In the present embodiment, the disinfection device 7a is described as a device used by the user for manually carrying out the disinfection. However, the disinfection device 7a may be a device configured to automatically carry out the disinfection.

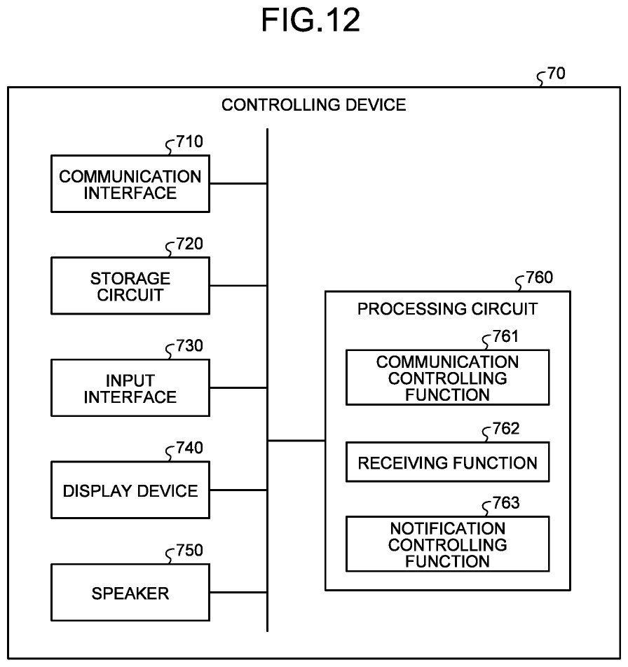

The disinfection device 7a includes the controlling device 70 configured to control the entirety of the disinfection device 7a. FIG. 12 is a block diagram illustrating an exemplary configuration of the controlling device 70 according to the second embodiment. As illustrated in FIG. 12, the controlling device 70 includes a communication interface circuit 710, a memory 720, an input interface circuit 730, a display 740, a speaker 750, and a processing circuit 760.

The communication interface circuit 710 is connected to the processing circuit 760 and is configured to control transfer of various types of data and communication between the medical information processing apparatus 1a and the controlling device 70. More specifically, the communication interface circuit 710 is configured to receive information from the medical information processing apparatus 1a and to output the received information to the processing circuit 760. For example, the communication interface circuit 710 is realized by using a network card, a network adaptor, an NIC or the like.

The memory 720 is connected to the processing circuit 760 and is configured to store therein various types of data. More specifically, the memory 720 is configured to store therein information about disinfection methods (e.g., types of disinfectant fluids that can be applied) which the disinfection device is capable of carrying out. Further, when the disinfection methods stored in the memory 720 are different from the disinfection methods determined in a disinfection plan, the medical information processing apparatus 1a may issue an alert for the user.

For example, the memory 720 is realized by using a semiconductor memory element such as a RAM or a flash memory, or a hard disk, an optical disk, or the like.

The input interface circuit 730 is connected to the processing circuit 760 and is configured to receive operations to input various types of instructions and various types of information from an operator. More specifically, the input interface circuit 730 is configured to convert the input operations received from the operator into electrical signals and to output the electrical signals to the processing circuit 760.

For example, the input interface circuit 730 is realized by using a trackball, a switch button, a mouse, a keyboard, a touchpad on which input operations can be performed by touching an operation surface thereof, a touch screen in which a display screen and a touchpad are integrally formed, a contactless input circuit using an optical sensor, an audio input circuit, and/or the like.

Further, the input interface circuit 730 does not necessarily have to include physical operation component parts such as a mouse, a keyboard, and/or the like. For instance, possible examples of the input interface circuit 730 include an electrical signal processing circuit configured to receive an electrical signal corresponding to an input operation from an external input device provided separately from the device and to output the electrical signal to a controlling circuit.

The display 740 is connected to the processing circuit 760 and is configured to display various types of information. More specifically, the display 740 is configured to convert data of the various types of information sent thereto from the processing circuit 760 into display-purpose electrical signals and to output the electrical signals. For example, the display 740 is realized by using a liquid crystal monitor, a touch panel, or the like.

The speaker 750 is connected to the processing circuit 760 and is configured to output sounds to the outside thereof according to control exercised by the processing circuit 760. More specifically, the speaker 750 is configured to convert data of various types of information sent thereto from the processing circuit 760 into output-purpose electrical signals and to output the electrical signals to the outside.

The processing circuit 760 is configured to control constituent elements of the controlling device 70 in accordance with the input operations received from the operator via the input interface circuit 730. More specifically, the processing circuit 760 is configured to exercise control so that an alert message is displayed on the display 140, according to a control signal received from the medical information processing apparatus 1a.

Further, the processing circuit 760 is configured to exercise control so that an alert sound is output from the speaker to the outside according to a control signal received from the medical information processing apparatus 1a. For example, the processing circuit 760 is realized by using a processor.

Further, the controlling device 70 includes various types of functions. More specifically, the controlling device 70 includes a communication controlling function 761, a receiving function 762, and a notification controlling function 763.

The communication controlling function 761 is configured to control communication between the medical information processing apparatus 1a and the controlling device 70. More specifically, the communication controlling function 761 is configured to transmit and receive data of various types of information to and from the medical information processing apparatus 1a.

The receiving function 762 is configured to receive various types of instructions from the user. More specifically, the receiving function 762 is configured to receive an instruction to start a disinfection process that is input by the user. Further, the receiving function 762 is configured to receive an instruction to end the disinfection process that is input by the user. In this situation, the instructions to start and end the disinfection process are input as a result of, for example, the user touching a start button and an end button displayed on the touch screen.

The various types of instructions received by the receiving function 762 are transmitted by the communication controlling function 761 to the medical information processing apparatus 1a as electrical signals (e.g., a start instruction signal, an end instruction signal, etc.).

The notification controlling function 763 is configured to control notifications for the user. More specifically, the notification controlling function 763 is configured to control the display 740 and the speaker 750 according to control signals transmitted from the medical information processing apparatus 1a, so as to display an alert message, to output an alert sound to the outside, and/or the like.

Alternatively, a part or all of the functional units of the controlling device 70 described above may be included in the user terminal 3.

Returning to the description of FIG. 11, the projector 8 is configured to project information output from the medical information processing apparatus 1a. The projector 8 is configured to project images on a screen, a wall, a floor, or the like, under control of the processing circuit 150 of the medical information processing apparatus 1a. Further, the projector 8 may project images on an arbitrary plane, object, space, or the like by implementing projection mapping. The medical information processing system Sa may include two or more projectors 8.

More specifically, the projector 8 is configured to project the 3D risk map generated by the medical information processing apparatus 1a, inside the medical facility.

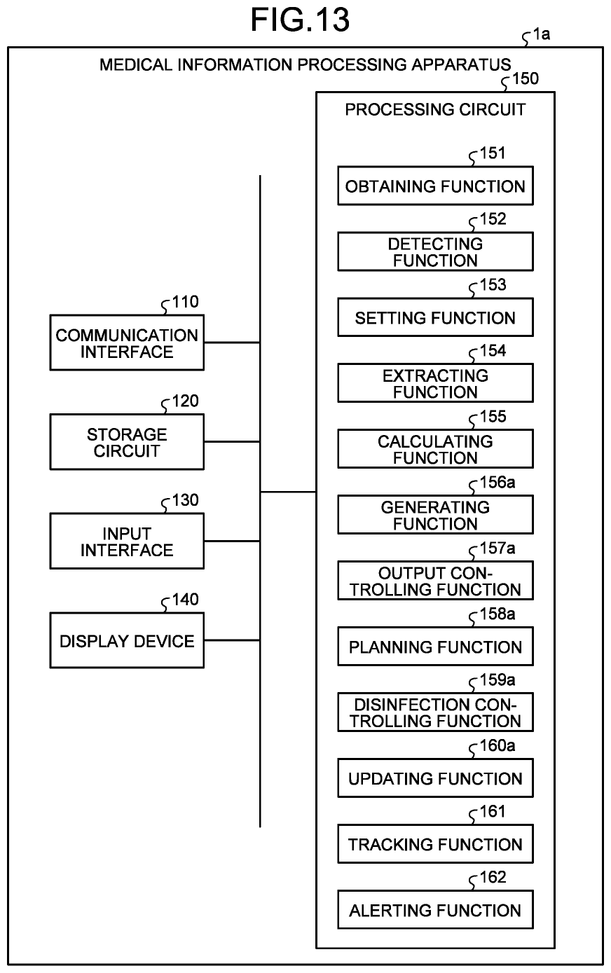

Next, the medical information processing apparatus 1a will be explained. FIG. 13 is a block diagram illustrating an exemplary configuration of the medical information processing apparatus 1a according to the second embodiment. As illustrated in FIG. 13, the medical information processing apparatus 1a according to the second embodiment includes, as functional units, the obtaining function 151, the detecting function 152, the setting function 153, the extracting function 154, the calculating function 155, a generating function 156a, an output controlling function 157a, a planning function 158a, a disinfection controlling function 159a, an updating function 160a, a tracking function 161, and an alerting function 162.

The generating function 156a is configured to generate a 3D risk map. More specifically, on the basis of data (e.g., 3D CAD data) indicating a three-dimensional structure of the medical facility, the generating function 156a is configured to generate the 3D risk map in which the risk values calculated by the calculating function 155 are indicated while being kept in association with three-dimensional positions in the medical facility. In this situation, the data indicating the three-dimensional structure of the medical facility is stored in the memory 120 or the like, for example.

The output controlling function 157a is configured to control the image projection by the projector 8. More specifically, the output controlling function 157a is configured to control the projector 8 so that the 3D risk map is projected in such a manner that the positions of structures in the 3D risk map match the actual positions of the structures in the medical facility. To control the projection of the 3D risk map, a publicly-known projection mapping technique may be used.

In this situation, possible output destinations of the 3D risk map are not limited to the projector 8. For example, the output controlling function 157a may be configured to control a display such as Augmented Reality (AR) glasses or the like, so that the 3D risk map is AR-displayed in such a manner that the positions of the structures in the 3D risk map match the actual positions of the structures in the medical facility, by using a publicly-known Augmented Reality (AR) technique.

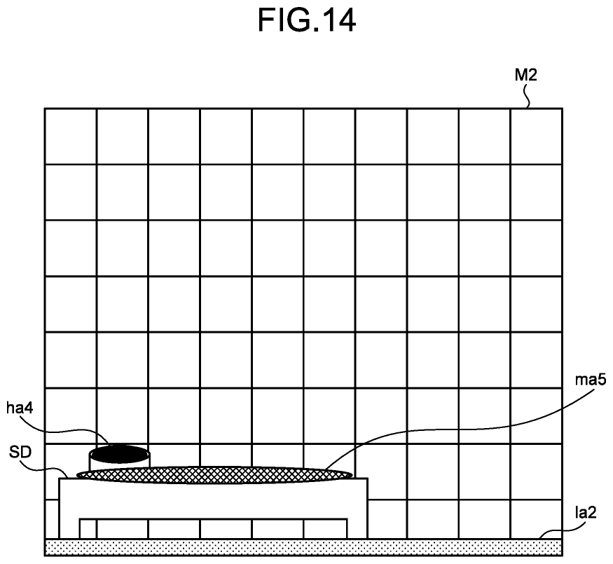

FIG. 14 is a drawing for explaining an example of the 3D risk map projecting process. In FIG. 14, a 3D risk map M2 indicating the vicinity of a consultation table in a consultation room is projected. More specifically, the output controlling function 157a is configured to control the projector 8 so that the 3D risk map M2 is projected by implementing projection mapping, in such a manner that various positions on a consultation table SD in the 3D risk map M2 match the actual positions on the consultation table.

In the example in FIG. 14, in the 3D risk map M2, an area having risk value 3 (an area ha4) is indicated with a solid black pattern, while another area having risk value 2 (an area ma5) is indicated with a hatching pattern, and yet another area having risk value 1 (an area la2) is indicated with a dot pattern. Similarly to the first embodiment, the area having risk value 3 may be projected together with information indicating what action was taken by the infected patient in the area.

Similarly to the first embodiment, the planning function 158a is configured to generate a disinfection plan. Additionally, in the second embodiment, the planning function 158a is configured to generate, together therewith, a disinfection path indicating a sequential order of disinfection in the 3D risk map. The disinfection path is projected inside the facility, together with the 3D risk map, under control of the output controlling function 157a. With this arrangement, the user is able to easily understand which area is to be disinfected next.

Further, as one element of the disinfection method, the planning function 158a is configured to determine a procedure (e.g., the disinfection application is to be carried out without changing the direction of the application) to be performed when the disinfection is manually carried out. If the user carries out the disinfection in a procedure different from the determined procedure, an alert is issued for the user under control of the alerting function 162 (explained later).

The disinfection controlling function 159a is configured to detect a state of the disinfection process manually performed by the user. More specifically, upon receipt of a reception signal instructing to start the disinfection process from the controlling device 70 of the disinfection device 7a, the disinfection controlling function 159a is configured to detect a start of the disinfection process. Upon detecting the start of the disinfection process, the tracking function 161 (explained later) is configured to start tracking movements of the disinfection device 7a. When the tracking of the movements of the disinfection device 7a is started, the disinfection controlling function 159a detects that the disinfection process is underway.

Further, upon receipt of a reception signal instructing to end the disinfection process from the controlling device 70 of the disinfection device 7a, when the risk values in the 3D risk map have each become a low risk value such as 0, the disinfection controlling function 159a is configured to detect an end of the disinfection process. On the contrary, when the risk values in the 3D risk map have not each become a low risk value such as 0, the disinfection controlling function 159a again detects that the disinfection process is underway.

The tracking function 161 is configured to track the movements of the disinfection device 7a held by the user. More specifically, by analyzing a plurality of images taken in a time series by the second cameras 6 and recognizing the positions of the disinfection device 7a rendered in the images on the basis of forms or the like, the tracking function 161 is configured to track the movements of the disinfection device 7a.

Possible methods for tracking the movements of the disinfection device 7a are not limited to the example described above. For instance, it is also acceptable to provide the disinfection device 7a with a sensor such as a pressure sensor or a Global Positioning System (GPS) sensor, so as to track the movements of the disinfection device 7a on the basis of sensing results from the sensor.

The alerting function 162 is configured to exercise control so as to issue an alert for the user. More specifically, the alerting function 162 is configured to transmit a control signal to the controlling device 70 of the disinfection device 7a via the communication interface circuit 110. The notification controlling function 763 of the controlling device 70 is configured to issue the alert for the user by controlling the display 740 and the speaker 750 according to the control signal.

For example, when it is detected that the user performed the disinfection process by using a wrong procedure, on the basis of a result of the tracking by the tracking function 161, the alerting function 162 is configured to transmit, to the controlling device 70, a control signal to have an alert message displayed. According to the control signal, the notification controlling function 763 of the controlling device 70 is configured to exercise control so as to cause the display 740 to display the alert message suggesting that the disinfection be carried out with the correct procedure.

In this situation, the alerting function 162 may transmit, to the controlling device 70, a control signal to have an alert sound output. In this situation, according to the control signal, the notification controlling function 763 is configured to exercise control so as to cause the speaker 750 to output the alert sound. The alerting function 162 may transmit, to the controlling device 70, one selected from between the control signal to have the alert message displayed and the control signal to have the alert sound output. Alternatively, both of the control signals may be transmitted to the controlling device 70 at the same time.

Further, the output controlling function 157a may exercise control so that an image indicating the correct procedure is projected inside the medical facility, at the same time as the alert is issued.

Further, when the disinfection controlling function 159a detects the start of the disinfection process, if the disinfection method which the disinfection device 7a is capable of carrying out and that is stored in the controlling device 70 is different from the disinfection method determined in the disinfection plan, the alerting function 162 may cause the notification controlling function 763 of the controlling device 70 to issue an alert suggesting that a proper disinfection device 7a be selected.

Further, when the risk values in the 3D risk map have not each become a low risk value such as 0, although the reception signal instructing to end the disinfection process is received from the controlling device 70 of the disinfection device 7a, the alerting function 162 may transmit a control signal to issue an alert for the user to indicate that the disinfection process is not completed.

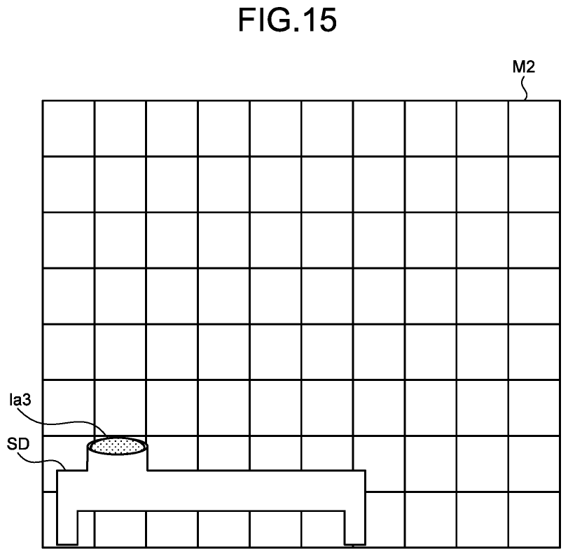

In this situation, for example, the output controlling function 157a may exercise control so as to project an image emphasizing an area of which the risk value in the 3D risk map has not become 0. Next, the control exercised to project the image emphasizing the area of which the risk value in the 3D risk map has not become 0 will be explained, with reference to FIG. 15.

FIG. 15 is a drawing for explaining another example of the 3D risk map projecting process. In FIG. 15, the 3D risk map M2 indicates that an area la3 has risk value 1. For example, let us discuss an example in which the 3D risk map M2 is in the state illustrated in FIG. 15 when a reception signal instructing to end the disinfection process is received from the controlling device 70 of the disinfection device 7a, the alerting function 162 is configured to transmit, to the controlling device 70 of the disinfection device 7a, a control signal to alert the user that the disinfection has not been completed.

Subsequently, the output controlling function 157a exercises control so that the places to be disinfected are displayed in an emphasized manner, by turning on and off the display of the area la3 in the 3D risk map M2 in FIG. 15, or the like. As a result, the user is able to easily understand which place in the medical facility has not completed the disinfection.

The updating function 160a is configured to update the 3D risk map. More specifically, on the basis of the result of the tracking by the tracking function 161, the updating function 160a is configured to perform a process of updating the risk values in the 3D risk map in a real-time manner. When the disinfection process by the user is completed after repeatedly performing the process described above, the updating function 160a updates the risk values of the places in the 3D risk map on which the disinfection process has been performed, with a low risk value such as 0.

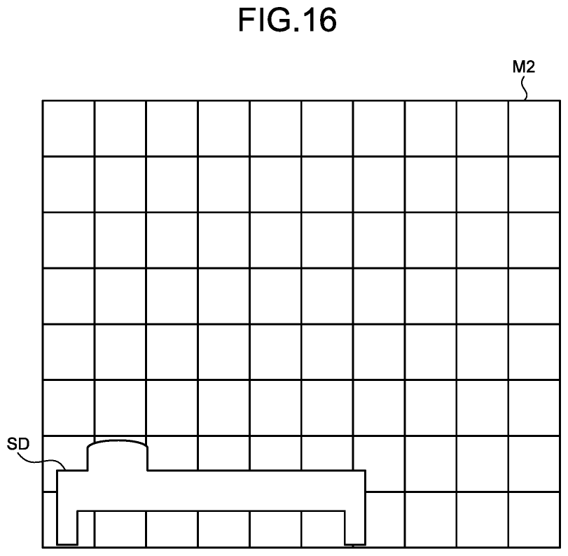

FIG. 16 is a drawing for explaining another example of the 3D risk map projecting process. For example, when the risk values in the 3D risk map M2 are in the state illustrated in FIG. 14 at the time of starting the disinfection, a transition is made to the state illustrated in FIG. 15 as a result of the user carrying out the disinfection process. Eventually, the state illustrated in FIG. 16 is achieved. In this situation, the 3D risk map M2 indicates that, as a result of the disinfection process, the risk values of the area la2 and the area ha4 (the area la3) illustrated in FIGS. 14 and 15 have each become 0.

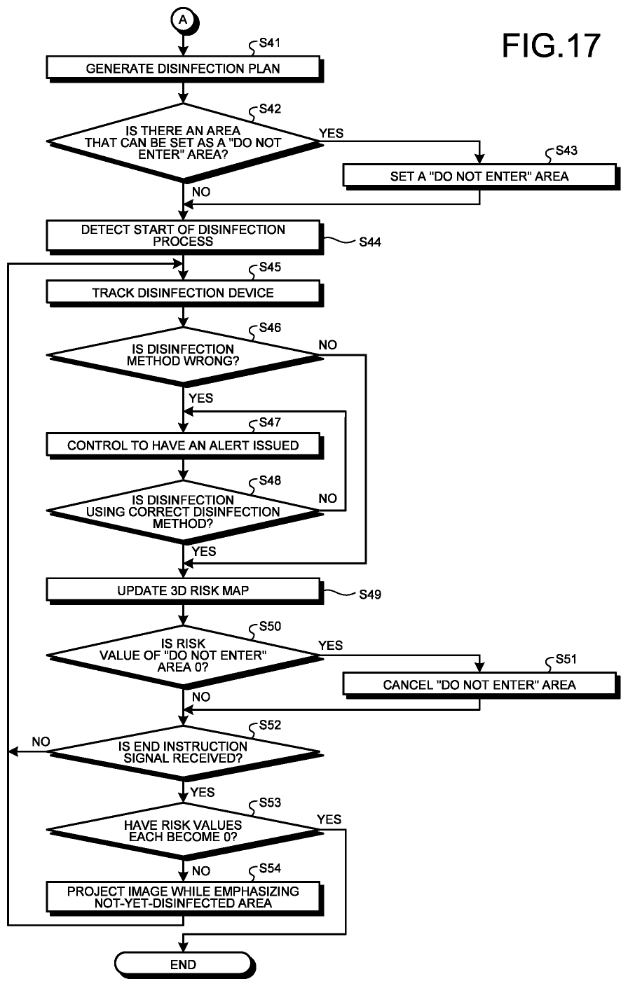

Next, a process performed by the medical information processing apparatus 1a according to the second embodiment will be explained, with reference to FIG. 17. FIG. 17 is a flowchart illustrating an example of a process related to the disinfection carried out by the medical information processing apparatus 1a according to the second embodiment. Because the risk map generating process is substantially the same as that in the first embodiment, explanations thereof will be omitted. It should be noted, however, that the generating function 156a is configured to generate a 3D risk map. Further, because the processes at steps S41 through S43 in FIG. 17 are the same as the processes at steps S21 through S23 in FIG. 9, explanations thereof will be omitted.

After the process at step S42 or step S43, the disinfection controlling function 159a detects a start of the disinfection process (step S44). More specifically, upon receipt of a start instruction signal from the controlling device 70 of the disinfection device 7a, the disinfection controlling function 159a detects the start of the disinfection process.

Subsequently, the tracking function 161 starts tracking movements of the disinfection device 7a (step S45). More specifically, the tracking function 161 tracks the movements of the disinfection device 7a on the basis of images taken in a time series by the second cameras 6. Subsequently, the tracking function 161 judges whether or not the user is carrying out the disinfection with the correct procedure (step S46). More specifically, on the basis of a result of the tracking and the disinfection procedure determined in the disinfection plan, the tracking function 161 judges whether or not the user is carrying out the disinfection with the correct procedure.

When the user is carrying out the disinfection with the correct procedure (step S46: Yes), the process proceeds to step S49. On the contrary, when the user is not carrying out the disinfection with the correct procedure (step S46: No), the alerting function 162 exercises control so as to cause the controlling device 70 of the disinfection device 7a to issue an alert (step S47).

For example, the alerting function 162 transmits, to the controlling device 70 of the disinfection device 7a, a control signal to cause the controlling device 70 to display an alert message. According to the control signal, the notification controlling function 763 of the controlling device 70 exercises control so as to cause the display 740 to display a message alerting that the disinfection should be carried out with the correct procedure.

Subsequently, similarly to step S46, the tracking function 161 judges whether or not the user is carrying out the disinfection with the correct procedure (step S48). When the user is carrying out the disinfection with the correct procedure (step S48: Yes), the process proceeds to step S49. On the contrary, when the user is not carrying out the disinfection with the correct procedure (step S48: No), the process proceeds to step S47.

Subsequently, the updating function 160a updates the 3D risk map (step S49). More specifically, the updating function 160a performs the process of decreasing the risk values in the 3D risk map, according to the result of the tracking by the tracking function 161. After that, among the areas of each of which the risk value in the 3D risk map has become a low risk value such as 0, the setting function 153 checks to see whether or not there is an area set as a Do Not Enter area (step S50).

When there is no area set as a Do Not Enter area (step S50: No), the process proceeds to step S52. When there is at least one area set as a Do Not Enter area (step S50: No), the setting function 153 cancels the setting of the Do Not Enter area (step S51). The cancellation of the setting as the Do Not Enter area is reflected in the 3D risk map by the updating function 160.

Subsequently, the disinfection controlling function 159a checks to see whether or not an end instruction signal is received from the controlling device 70 of the disinfection device 7a (step S52). When no end instruction signal is received (step S52: No), the process proceeds to step S45. On the contrary, when an end instruction signal is received (step S52: Yes), the disinfection controlling function 159a checks to see whether or not the risk values in the 3D risk map have each become a low risk value such as 0 (step S53).

When the risk values have not each become a low risk value such as 0 (step S53: No), the output controlling function 157a exercises control so as to project an image emphasizing one or more not-yet-disinfected areas (the areas of each of which the risk value in the 3D risk map has not become a low risk value such as 0) (step S54). After that, the process proceeds to step S45. On the contrary, when the risk values have each become a low risk value such as 0 (step S53: Yes), the present process is ended.

The medical information processing apparatus 1a according to the second embodiment described above is configured to generate the 3D risk map and to exercise control so as to project the 3D risk map inside the medical facility by controlling the projector 8. As a result, the user is able to easily understand the areas in the medical facility that need to be disinfected.

The first embodiment and the second embodiment described above may be implemented while being modified as appropriate, by changing a part of the constituent elements or the functions of the apparatuses and/or the devices. Thus, in the following sections, a number of modification examples related to the above embodiments will be explained as other embodiments. In the following sections, differences from the first and the second embodiments presented above will primarily be explained, and detailed explanations of certain aspects that have already been explained will be omitted. Further, the modification examples described below may be carried out individually or may be carried out in combination as appropriate.

### First Modification Example

In the first embodiment and the second embodiment described above, the generating function 156 (156a) is configured to generate the risk map indicating the risk values of the various positions in the facility, as well as the details of the actions taken in the higher risk areas by the patient found to have contracted the infectious disease; however, possible modes of the risk map generated by the generating function 156 (156a) are not limited to this example.

For example, in addition to the configuration above, the generating function 156 may be configured to generate a risk map indicating, within the map, the patient ID of the patient found to have contracted the infectious disease or patient IDs of close-contact people.

According to the present modification example, in the situation where there are two or more close-contact people, it is possible to easily understand the actions taken by each of the patients.

### Second Modification Example

In the first embodiment and the second embodiment described above, the extracting function 154 is configured to extract the actions of the patient by determining that the patient contracted the infectious disease when the test result related to the infectious disease performed in the medical facility is positive; however, possible examples of the process performed by the extracting function 154 to extract the actions of the patient with respect to the patient determined to have contracted the infectious disease are not limited to this example.

For instance, the extracting function 154 may be configured to extract the actions of the patient when having received, from an external medical institution or the like, information about the patient found to have contracted the infectious disease.

More specifically, when having received, from the external medical institution or the like, patient identification information such as a face image capable of identifying the patient who contracted the infectious disease, the extracting function 154 may be configured to extract the actions of the patient from the action history information on the basis of the patient information identification information.

According to the present modification example, for instance, it is possible to immediately understand the actions of the patient in the facility even in the situation where, for example, a test result from the facility was negative while no prior information was available, but information indicating a positive result of a test performed later at another facility was received.

### Third Modification Example

In the first embodiment and the second embodiment described above, the medical information processing apparatus 1 (1a) is provided in the medical facility such as a hospital. However, the medical information processing apparatus 1 may be provided in a place other than medical facilities. More specifically, for example, the place where the medical information processing apparatus 1 is provided may be a facility such as an airport, a school, or the like.

When the tests for the infectious disease are not performed in the facility in question, the medical information processing apparatus 1 (1a) may receive test results of the infectious disease from a medical institution. Further, the medical information processing apparatus 1 (1a) may determine that a patient contracted the infectious disease on the basis of, for example, self-reporting from a student, a staff member, or the like.

### Fourth Modification Example

In the first embodiment and the second embodiment described above, the calculating function 155 is configured to calculate the risk values of the various positions, on the basis of the patient having the positive test result related to the infectious disease and the actions of the patient; however, possible modes of the risk values calculated by the calculating function 155 are not limited to this example.

For example, the calculating function 155 may be configured to calculate risk values of the various positions in accordance with the number of people or a degree of concentration of people.

According to the present modification example, because there is no need to extract the actions of the patient, it is possible to reduce processing loads imposed on the processing circuit 15.

### Fifth Modification Example

In the first embodiment and the second embodiment described above, the calculating function 155 is configured to calculate the risk values of the various positions, by extracting the actions of the patient from the images inside the facility taken by the plurality of second cameras 6. However, possible modes of the risk values calculated by the calculating function 155 are not limited to this example.

For instance, the calculating function 155 may be configured to calculate the risk values of the various positions, in accordance with detection results of a temperature sensor, a radar, or the like provided in the facility.

According to the present modification example, because there is no need to take the images including the face image, it is possible to calculate the risk values without invading privacy.

According to at least one aspect of the embodiments described above, it is possible to understand the actions taken in the facility by the patient found to have contracted the infectious disease.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising a processing circuitry configured:
to obtain, from each of patients entering a facility, identification information capable of identifying the patient;
to extract, when it is discovered that at least one of the patients has contracted a predetermined disease, an action taken in each of positions in the facility by a target patient who has contracted the disease, from action history information recording actions taken in the facility by each of the patients identified with the identification information, on a basis of the identification information of the target patient;
to calculate, with respect to each of the positions, a first risk value of a risk imposed by the disease on a surrounding environment, on a basis of the extracted actions of the target patient;
to generate a risk map indicating the calculated first risk values so as to be kept in association with the positions in the facility; and
to exercise control so as to output the generated risk map.

2. The medical information processing apparatus according to claim 1, wherein the processing circuitry generates the risk map in which each of the first risk values of the positions is indicated in a corresponding position within a map representing the facility.

3. The medical information processing apparatus according to claim 1, wherein the processing circuitry generates the risk map in which the action taken by the target patient in each of the positions in the facility is indicated in a corresponding position within a map representing the facility.

4. The medical information processing apparatus according to claim 1, wherein the processing circuitry obtains the identification information indicating an appearance characteristic of the patient, from an image taken of the patient.

5. The medical information processing apparatus according to claim 1, wherein the processing circuitry obtains an image taken of an inside of the facility as the action history information, from one or more imaging devices configured to image the positions in the facility.

6. The medical information processing apparatus according to claim 1, wherein
on a basis of a detection result from a sensor configured to detect states of the patients, the processing circuitry detects a patient predicted to have a high possibility of having contracted the disease, and
upon detection of the patient predicted to have the high possibility of having contracted the disease, the processing circuitry outputs a notification to notify a user of the detection.

7. The medical information processing apparatus according to claim 1, wherein, on a basis of the generated risk map, the processing circuitry generates a disinfection plan determining a disinfection method for each of the positions in the facility.

8. The medical information processing apparatus according to claim 7, wherein the processing circuitry judges whether or not disinfection is required with respect to each of the positions in the facility.

9. The medical information processing apparatus according to claim 8, wherein, with respect to each of positions in the facility for which the disinfection is determined to be required, the processing circuitry generates the disinfection plan determining a disinfection method, a disinfection time period, and a sequential order of disinfection.

10. The medical information processing apparatus according to claim 7, wherein the processing circuitry carries out disinfection according to the generated disinfection plan.

11. The medical information processing apparatus according to claim 10, wherein the processing circuitry updates the generated risk map in conjunction with the disinfection.

12. The medical information processing apparatus according to claim 11, wherein the processing circuitry updates the generated risk map in conjunction with the disinfection.

13. The medical information processing apparatus according to claim 1, wherein
when there is a contact person being a patient who was in contact with the target patient, the processing circuitry obtains the identification information capable of identifying the contact person,
the processing circuitry extracts an action taken in each of positions in the facility by the contact person, from the action history information, and
the processing circuitry calculates the first risk value with respect to each of the positions, on a basis of the extracted actions of the contact person.

14. The medical information processing apparatus according to claim 13, wherein the processing circuitry calculates a second risk value of a risk imposed by the disease on the contact person, on a basis of the extracted actions of the target patient and the contact person.

15. The medical information processing apparatus according to claim 14, wherein the processing circuitry calculates the first risk value on a basis of the extracted actions of the contact person and the calculated second risk value.

16. The medical information processing apparatus according to claim 1, wherein
the processing circuitry generates the risk map that is three-dimensional, and
the processing circuitry outputs the generated risk map to a projector and controls the projector so as to project the risk map inside the facility.
